(19) United States
(12) Patent Application Publication
(10) Pub. No.: US 2001/0028437 A1
(43) Pub. Date: Oct. 11, 2001

(54) System for exercising eye muscles

(76) Inventors: Steven M. Beresford, Vancovver, WA (US); David W. Muris, Sacramento, CA (US); Merrill J. Allen, Bloomington, IN (US); Francis A. Young, Pullman, WA (US)

Correspondence Address: VENABLE, Post Office Box 34385, Washington, DC, 20043-9998, US

(21) Appl. No.: 09/741,091

(22) Filed: Dec. 21, 2000

Related U.S. Application Data
(60) Provisional application No. 60/171,513, filed on Dec. 22, 1999.
(60) Provisional application No. 60/174,881, filed on Jan. 7, 2000.

(51) Int. Cl. A61B 3/00

(52) U.S. Cl. 351/203

(57) ABSTRACT

The invention includes a method for exercising muscles in and around the eye by performing exercises that maintain and improve vision and the function of the eye. The invention includes individual general, timed and ordered sequences of exercises for exercising the eye muscles. The sequences that are preferably performed on a rotating basis. The invention also includes a kit providing a presentation of the exercise steps and sequences.

### CROSS-REFERENCE TO RELATED APPLICATIONS

This application claims the priority of U.S. Provisional Patent Application Nos. 60/171,513 and Ser. No. 60/174,881, filed Dec. 22, 1999 and Jan. 7, 2000, respectively, which are incorporated herein by reference in their entirety.

### BACKGROUND OF THE INVENTION

This invention relates to a method for exercising the muscles in and around the eye. Specifically, the invention is a method for exercising the muscles in and around the eye by performing a set or sets of exercise sequences.

Treatment for eye problems is frequently accomplished by use of “corrective” lenses or refractive surgery. Corrective lenses and refractive surgery allow the user to see more clearly, but do not improve the natural focusing power of the eye or prevent further degeneration of vision. By exercising the eye, degeneration of vision can be halted or even reversed with a resultant improvement in vision and reduction of discomfort, such as eye strain, computer-related visual stress such as Computer Vision Syndrome, or visual stress and other eye problems related to computer use. See, for example, the book authored by the inventors: “Improve Your Vision Without Glasses or Contact Lenses: A New Program of Therapeutic Eye Exercises” by Drs. Steven M. Beresford, David W. Muris, Merrill J. Allen, Francis A. Young (Simon & Schuster, 1996), which is incorporated herein by reference in its entirety.

### SUMMARY OF THE INVENTION

The inventors' book provides ten example sequences of eye exercises directed at stabilizing or improving myopia, astigmatism, presbyopia, hyperopia, dry eye syndrome, cataract and macular degeneration. However, no general program of eye exercise routines was presented, and the reader was essentially left to develop his or her own program of eye exercise routines. The present invention overcomes this deficiency by presenting a program of eye exercise sequences to maintain and improve vision.

The invention includes a method for exercising muscles in and around the eye by performing exercises that maintain and improve vision and the function of the eye.

In one embodiment, the invention includes a method of practicing at least one of seven individual general sequences for exercising the eye muscles. In another aspect, the invention includes a method of practicing at least one of seven individual timed sequences for exercising the eye muscles. In yet another aspect, the invention includes a method of practicing at least one of seven individual ordered sequences for exercising the eye muscles. In another aspect, the invention includes three or four of the general sequences performed on a rotating basis. In another aspect, the invention includes three or four of the timed sequences performed on a rotating basis. In another aspect, the invention includes three or four of the ordered sequences performed on a rotating basis.

In another embodiment of the invention, an eye patch is worn daily after completion of the exercise sequences to improve vision.

In yet another embodiment of the invention, a kit includes a presentation of at least one sequence of the invention. The presentation can be embodied in, for example, one or more of each of the following: a printed instruction manual, a videotape, a chart, a journal and an audio tape. As an option, the presentation can be embodied in a computer-readable medium.

In yet another embodiment, the invention includes accessing a web site on the Internet and obtaining additional exercise sequences. The additional exercise sequences may be, for example, additional exercises for further improving vision or maintaining vision quality.

In another aspect, the invention is a computer system having a computer-readable medium with software including software for presenting instructions for performing one or more of the individual eye exercises that comprise the sequences of the invention. Preferably, the software includes software for presenting one or more of the sequences of the invention. The software may also include software for modifying the sequences and presenting instructions for performing the modified sequences. In a preferred embodiment, the software includes sequences chosen to treat or prevent Computer Vision Syndrome and may also include software presenting instructions for performing visual habits. In particularly preferred embodiments, the software presents a reminder to perform an exercise sequence and/or visual habit, preferably at approximately periodic intervals.

Further objectives and advantages will become apparent from a consideration of the description herein.

### BRIEF DESCRIPTION OF THE DRAWINGS

The invention is better understood by reading the following detailed description with reference to the accompanying figures, in which like reference numerals refer to like elements throughout, and in which:

FIGS. 1A and 1B illustrate the Acupressure Variation C.

FIG. 2 is a Fusion Chart for use with some steps of practicing the invention.

FIG. 3 is a Word Chart for use with some steps of practicing the invention.

FIG. 4 is a Scanning Chart for use with some steps of practicing the invention.

FIG. 5 is a block diagram of the computer system of the invention.

### DETAILED DESCRIPTION OF INVENTION

In describing preferred embodiments of the present invention, specific terminology is employed for the sake of clarity. However, the invention is not intended to be limited to the specific terminology so selected.

Subsequent to the publication of the book by the inventors, the inventors conducted additional research to develop programs of eye exercise sequences that could be used generally to strengthen and tone the muscles in and around the eye, improve coordination of the eye muscles, and improve the flow of blood and other fluids in and around the eyes, which is applicable to a broad range of users. It was found that particular sequences were preferable for obtaining successful results and not causing undue fatigue. With the invention, the flow of blood and other fluids in and around the eyes can be improved, in order to promote ocular health, and reduce the risk of degenerative eye diseases, such as, for example, cataract, macular or retinal degeneration, glaucoma, and dry eye syndrome.

The invention is also useful for the prevention and treatment of Computer Vision Syndrome. “Computer Vision Syndrome” refers to a condition of fatigue of the muscles in and around the eye. Symptoms of Computer Vision Syndrome may include: tired, dry, achy, itchy, gritty, or sore eyes; double or blurred vision; loss of distance vision; ocular fatigue; and tics or spasms in the eyelids and muscles surrounding the eyes. Additional symptoms may include: headaches; dizziness; confusion; disorientation; and feelings of being “stressed out,” overloaded or overwhelmed. Computer Vision Syndrome commonly results from prolonged viewing objects of which are relatively close to the eye, for example from spending significant time viewing a computer screen while typing or inputting data into the computer. Although this is the most common cause of Computer Vision Syndrome, it will be appreciated that any similar work which has a similar fatiguing effect on the muscles in and around the eye may also lead to the occurrence of Computer Vision Syndrome.

Although the exercise sequences of the present invention are unique, the individual exercises that make up the sequences of the method are described in the inventors' book. The previously published book includes Acupressure exercises described as Acupressure A, Acupressure B, and Acupressure C which correspond to the exercises denoted herein as Acupressure Variations A, D, and B, respectively.

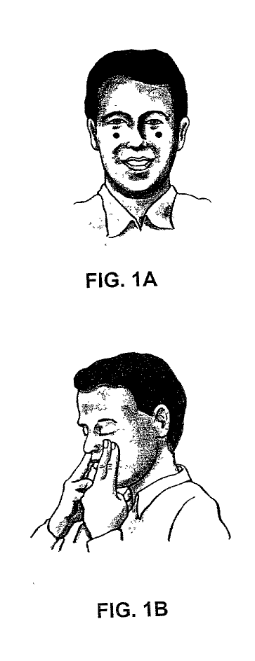

FIGS. 1A and 1B illustrate the Acupressure Variation C. which is utilized in the present invention. Acupressure Variation C is practiced by placing two or three fingers on the acupressure point on the crest of the cheekbones as indicated in FIG. 1A. The fingers are placed on these points as shown in FIG. 1B. With closed eyes, the crest of the cheekbones is massaged by moving the skin in circles with the two or three fingers. Advantageously, the direction of rotations is changed approximately every few seconds.

The invention utilizes the following seven independent general sequences of exercises:

General Sequence A:

Exercising at least one eye with a Hydrotherapy exercise;

exercising at least one eye with Clock Rotations;

exercising at least one eye with Eye Rolls;

exercising at least one eye with Pumping;

exercising at least one eye with Squeeze Blinking;

exercising at least one eye with Slow Blinking;

exercising at least one eye with Fast Blinking;

exercising at least one eye with a Acupressure Variation A;

exercising at least one eye with a Acupressure Variation B;

exercising at least one eye with Scanning using a Scanning Chart;

exercising at least one eye with a Acupressure Variation C;

exercising at least one eye with a Acupressure Variation D;

exercising at least one eye with Clock Rotations;

exercising at least one eye with Eye Rolls;

exercising at least one eye with Pumping;

exercising at least one eye with Light Therapy; and

exercising at least one eye with Palming.

General Sequence B:

Exercising at least one eye with Tromboning;

exercising at least one eye with Nose Fusion;

exercising at least one eye with using Fusion Chart;

exercising at least one eye with Fusion Pumping;

exercising at least one eye with using Word Chart;

exercising at least one eye with Blur Reading;

exercising at least one eye with Scanning using Scanning Chart;

exercising at least one eye with Blur Zoning;

exercising at least one eye with using Word Chart;

exercising at least one eye with Tromboning;

exercising at least one eye with Nose Fusion;

exercising at least one eye with using Fusion Chart;

exercising at least one eye with Fusion Pumping;

exercising at least one eye with Light Therapy; and

exercising at least one eye with Palming.

General Sequence C:

Exercising at least one eye with Squeeze Blinking;

exercising at least one eye with Slow Blinking;

exercising at least one eye with Fast Blinking;

exercising at least one eye with Clock Rotations;

exercising at least one eye with Fusion Pumping;

exercising at least one eye with Light Therapy;

exercising at least one eye with Palming;

exercising at least one eye with using Word Chart;

exercising at least one eye with Squeeze Blinking;

exercising at least one eye with Slow Blinking;

exercising at least one eye with Fast Blinking;

exercising at least one eye with Clock Rotations;

exercising at least one eye with Fusion Pumping;

exercising at least one eye with Light Therapy;

exercising at least one eye with Palming; and

exercising at least one eye with using Word Chart.

General Sequence D:

Exercising at least one eye with Pumping;

exercising at least one eye with Clock Rotations;

exercising at least one eye with Tromboning;

exercising at least one eye with Eye Rolls;

exercising at least one eye with Pumping;

exercising at least one eye with Squeeze Blinking;

exercising at least one eye with Slow Blinking;

exercising at least one eye with Fast Blinking;

exercising at least one eye with Pumping;

exercising at least one eye with Clock Rotations;

exercising at least one eye with Tromboning;

exercising at least one eye with Eye Rolls;

exercising at least one eye with Pumping; and

exercising at least one eye with Hydrotherapy.

General Sequence E

Exercising at least one eye with Slow Blinking;

exercising at least one eye with Squeeze Blinking;

exercising at least one eye with Blur Zoning/Edging; and

exercising at least one eye with Blur Zoning/Centering.

General Sequence F

Exercising at least one eye with Acupressure Variation A;

exercising at least one eye with Acupressure Variation B;

exercising at least one eye with Acupressure Variation C; and

exercising at least one eye with Acupressure Variation D.

General Sequence G

Exercising at least one eye with Eye Rolls;

exercising at least one eye with Clock Rotations;

exercising at least one eye with Pumping; and

exercising at least one eye with Tromboning.

In some embodiments, the invention utilizes the following seven independent timed sequences of exercises:

Timed Sequence H:

Exercising at least one eye with a Hydrotherapy exercise for approximately three minutes;

exercising at least one eye with Clock Rotations for approximately two minutes;

exercising at least one eye with Eye Rolls for approximately one minute;

exercising at least one eye with Pumping for approximately three minutes;

exercising at least one eye with Squeeze Blinking for approximately one minute;

exercising at least one eye with Slow Blinking for approximately one minute;

exercising at least one eye with Fast Blinking for approximately one minute;

exercising at least one eye with a Acupressure Variation A for approximately one minute;

exercising at least one eye with a Acupressure Variation B for approximately one minute;

exercising at least one eye with Scanning using a Scanning Chart for approximately two minutes;

exercising at least one eye with a Acupressure Variation C for approximately one minute;

exercising at least one eye with a Acupressure Variation D for approximately one minute;

exercising at least one eye with Clock Rotations for approximately two minutes;

exercising at least one eye with Eye Rolls for approximately one minute;

exercising at least one eye with Pumping for approximately three minutes;

exercising at least one eye with Light Therapy for approximately three minutes; and

exercising at least one eye with Palming for approximately three minutes.

Timed Sequence I:

Exercising at least one eye with approximately three minutes of Tromboning;

exercising at least one eye with approximately one minute of Nose Fusion;

exercising at least one eye with approximately two minutes of using Fusion Chart;

exercising at least one eye with approximately two minutes of Fusion Pumping;

exercising at least one eye with approximately two minutes of using Word Chart;

exercising at least one eye with approximately one minute of Blur Reading;

exercising at least one eye with approximately two minutes of Scanning using Scanning Chart;

exercising at least one eye with approximately one minute of Blur Zoning;

exercising at least one eye with approximately two minutes of using Word Chart;

exercising at least one eye with approximately three minutes of Tromboning;

exercising at least one eye with approximately one minute of Nose Fusion;

exercising at least one eye with approximately two minutes of using Fusion Chart;

exercising at least one eye with approximately two minutes of Fusion Pumping;

exercising at least one eye with approximately three minutes of Light Therapy; and

exercising at least one eye with approximately three minutes of Palming.

Timed Sequence J:

Exercising at least one eye with approximately one minute of Squeeze Blinking;

exercising at least one eye with approximately one minute of Slow Blinking;

exercising at least one eye with approximately one minute of Fast Blinking;

exercising at least one eye with approximately two minutes of Clock Rotations;

exercising at least one eye with approximately two minutes of Fusion Pumping;

exercising at least one eye with approximately three minutes of Light Therapy;

exercising at least one eye with approximately three minutes of Palming;

exercising at least one eye with approximately two minutes of using Word Chart;

exercising at least one eye with approximately one minute of Squeeze Blinking;

exercising at least one eye with approximately one minute of Slow Blinking;

exercising at least one eye with approximately one minute of Fast Blinking;

exercising at least one eye with approximately two minutes of Clock Rotations;

exercising at least one eye with approximately two minutes of Fusion Pumping;

exercising at least one eye with approximately three minutes of Light Therapy;

exercising at least one eye with approximately three minutes of Palming; and

exercising at least one eye with approximately two minutes of using Word Chart.

Timed Sequence K:

Exercising at least one eye with approximately three minutes of Pumping;

exercising at least one eye with approximately two minutes of Clock Rotations;

exercising at least one eye with approximately three minutes of Tromboning;

exercising at least one eye with approximately one minute of Eye Rolls;

exercising at least one eye with approximately three minutes of Pumping;

exercising at least one eye with approximately one minute of Squeeze Blinking;

exercising at least one eye with approximately one minute of Slow Blinking;

exercising at least one eye with approximately one minute of Fast Blinking;

exercising at least one eye with approximately three minutes of Pumping;

exercising at least one eye with approximately two minutes of Clock Rotations;

exercising at least one eye with approximately three minutes of Tromboning;

exercising at least one eye with approximately one minute of Eye Rolls;

exercising at least one eye with approximately three minutes of Pumping; and

exercising at least one eye with approximately three minutes of Hydrotherapy.

Timed Sequence L:

Exercising at least one eye with Slow Blinking for approximately forty seconds;

exercising at least one eye with Squeeze Blinking for approximately forty seconds;

exercising at least one eye with Blur Zoning/Edging for approximately forty seconds; and

exercising at least one eye with Blur Zoning/Centering for approximately forty seconds.

Timed Sequence M:

Exercising at least one eye with Acupressure Variation A for approximately forty seconds;

exercising at least one eye with Acupressure Variation B for approximately forty seconds;

exercising at least one eye with Acupressure Variation C for approximately forty seconds; and

exercising at least one eye with Acupressure Variation D for approximately forty seconds.

Timed Sequence N:

Exercising at least one eye with Eye Rolls for approximately forty seconds;

exercising at least one eye with Clock Rotations for approximately forty seconds;

exercising at least one eye with Pumping for approximately forty seconds; and

exercising at least one eye with Tromboning for approximately forty seconds.

In preferred embodiments, the invention utilizes the following seven independent ordered sequences of exercises:

Ordered Sequence O:

Exercising at least one eye with a Hydrotherapy exercise for approximately three minutes; followed by

exercising at least one eye with Clock Rotations for approximately two minutes; followed by

exercising at least one eye with Eye Rolls for approximately one minute; followed by

exercising at least one eye with Pumping for approximately three minutes; followed by

exercising at least one eye with Squeeze Blinking for approximately one minute; followed by

exercising at least one eye with Slow Blinking for approximately one minute; followed by

exercising at least one eye with Fast Blinking for approximately one minute; followed by

exercising at least one eye with a Acupressure Variation A for approximately one minute; followed by

exercising at least one eye with a Acupressure Variation B for approximately one minute; followed by

exercising at least one eye with Scanning using a Scanning Chart for approximately two minutes; followed by

exercising at least one eye with a Acupressure Variation C for approximately one minute; followed by

exercising at least one eye with a Acupressure Variation D for approximately one minute; followed by

exercising at least one eye with Clock Rotations for approximately two minutes; followed by

exercising at least one eye with Eye Rolls for approximately one minute; followed by

exercising at least one eye with Pumping for approximately three minutes; followed by

exercising at least one eye with Light Therapy for approximately three minutes; followed by

exercising at least one eye with Palming for approximately three minutes.

Ordered Sequence P:

Exercising at least one eye with approximately three minutes of Tromboning; followed by

exercising at least one eye with approximately one minute of Nose Fusion; followed by

exercising at least one eye with approximately two minutes of using Fusion Chart; followed by

exercising at least one eye with approximately two minutes of Fusion Pumping; followed by

exercising at least one eye with approximately two minutes of using Word Chart; followed by

exercising at least one eye with approximately one minute of Blur Reading; followed by

exercising at least one eye with approximately two minutes of Scanning using Scanning Chart; followed by

exercising at least one eye with approximately one minute of Blur Zoning; followed by

exercising at least one eye with approximately two minutes of using Word Chart; followed by

exercising at least one eye with approximately three minutes of Tromboning; followed by

exercising at least one eye with approximately one minute of Nose Fusion; followed by

exercising at least one eye with approximately two minutes of using Fusion Chart; followed by

exercising at least one eye with approximately two minutes of Fusion Pumping; followed by

exercising at least one eye with approximately three minutes of Light Therapy; followed by

exercising at least one eye with approximately three minutes of Palming.

Ordered Sequence Q:

Exercising at least one eye with approximately one minute of Squeeze Blinking; followed by

exercising at least one eye with approximately one minute of Slow Blinking; followed by

exercising at least one eye with approximately one minute of Fast Blinking; followed by

exercising at least one eye with approximately two minutes of Clock Rotations; followed by

exercising at least one eye with approximately two minutes of Fusion Pumping; followed by

exercising at least one eye with approximately three minutes of Light Therapy; followed by

exercising at least one eye with approximately three minutes of Palming; followed by

exercising at least one eye with approximately two minutes of using Word Chart; followed by

exercising at least one eye with approximately one minute of Squeeze Blinking; followed by

exercising at least one eye with approximately one minute of Slow Blinking; followed by

exercising at least one eye with approximately one minute of Fast Blinking; followed by

exercising at least one eye with approximately two minutes of Clock Rotations; followed by

exercising at least one eye with approximately two minutes of Fusion Pumping; followed by

exercising at least one eye with approximately three minutes of Light Therapy; followed by

exercising at least one eye with approximately three minutes of Palming; followed by

exercising at least one eye with approximately two minutes of using Word Chart.

Ordered Sequence R:

Exercising at least one eye with approximately three minutes of Pumping; followed by

exercising at least one eye with approximately two minutes of Clock Rotations; followed by

exercising at least one eye with approximately three minutes of Tromboning; followed by

exercising at least one eye with approximately one minute of Eye Rolls; followed by

exercising at least one eye with approximately three minutes of Pumping; followed by

exercising at least one eye with approximately one minute of Squeeze Blinking; followed by

exercising at least one eye with approximately one minute of Slow Blinking; followed by

exercising at least one eye with approximately one minute of Fast Blinking; followed by

exercising at least one eye with approximately three minutes of Pumping; followed by

exercising at least one eye with approximately two minutes of Clock Rotations; followed by

exercising at least one eye with approximately three minutes of Tromboning; followed by

exercising at least one eye with approximately one minute of Eye Rolls; followed by

exercising at least one eye with approximately three minutes of Pumping; followed by

exercising at least one eye with approximately three minutes of Hydrotherapy.

Ordered Sequence S:

Exercising at least one eye with Slow Blinking for approximately forty second; followed by

exercising at least one eye with Squeeze Blinking for approximately forty seconds; followed by

exercising at least one eye with Blur Zoning/Edging for approximately forty seconds; and followed by

exercising at least one eye with Blur Zoning/Centering for approximately forty seconds.

Ordered Sequence T:

Exercising at least one eye with Acupressure Variation A for approximately forty seconds; followed by

exercising at least one eye with Acupressure Variation B for approximately forty seconds; followed by

exercising at least one eye with Acupressure Variation C for approximately forty seconds; followed by

exercising at least one eye with Acupressure Variation D for approximately forty seconds.

Ordered Sequence U:

Exercising at least one eye with Eye Rolls for approximately forty seconds; followed by

exercising at least one eye with Clock Rotations for approximately forty seconds; followed by

exercising at least one eye with Pumping for approximately forty seconds; followed by

exercising at least one eye with Tromboning for approximately forty seconds.

General Sequence E, Timed Sequence L, and Ordered Sequence S may be referred to as eye relaxation sets. General Sequence F, Timed Sequence M and Ordered Sequence T may be referred to as eye massage sets. General Sequence G, Timed Sequence N and Ordered Sequence U may be referred to as power vision sets.

Hereafter, the term “sequence,” if used without specifying a particular sequence above, shall mean any of General Sequences A through G, Timed Sequences H through N, or Ordered Sequences O through U. Times are approximate for all exercises.

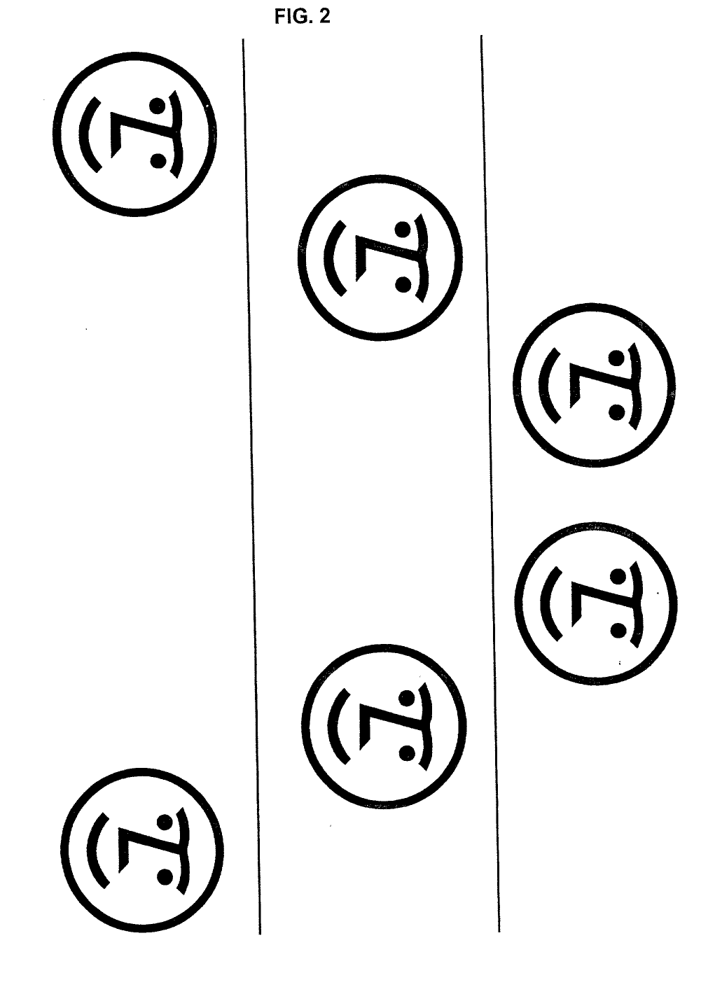

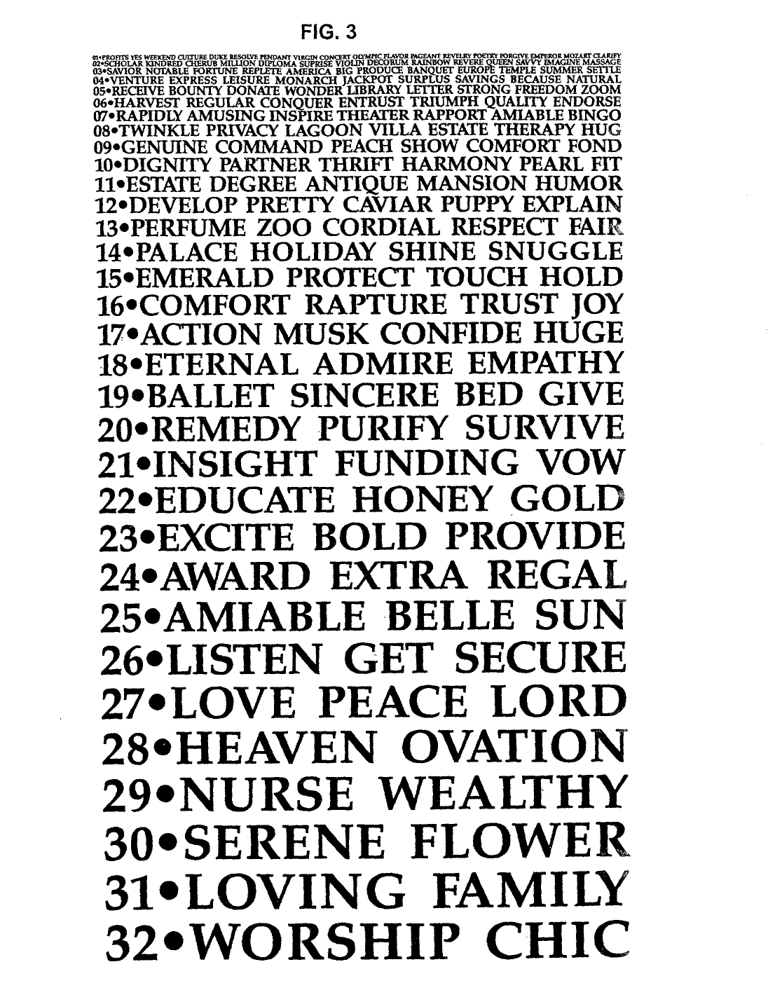

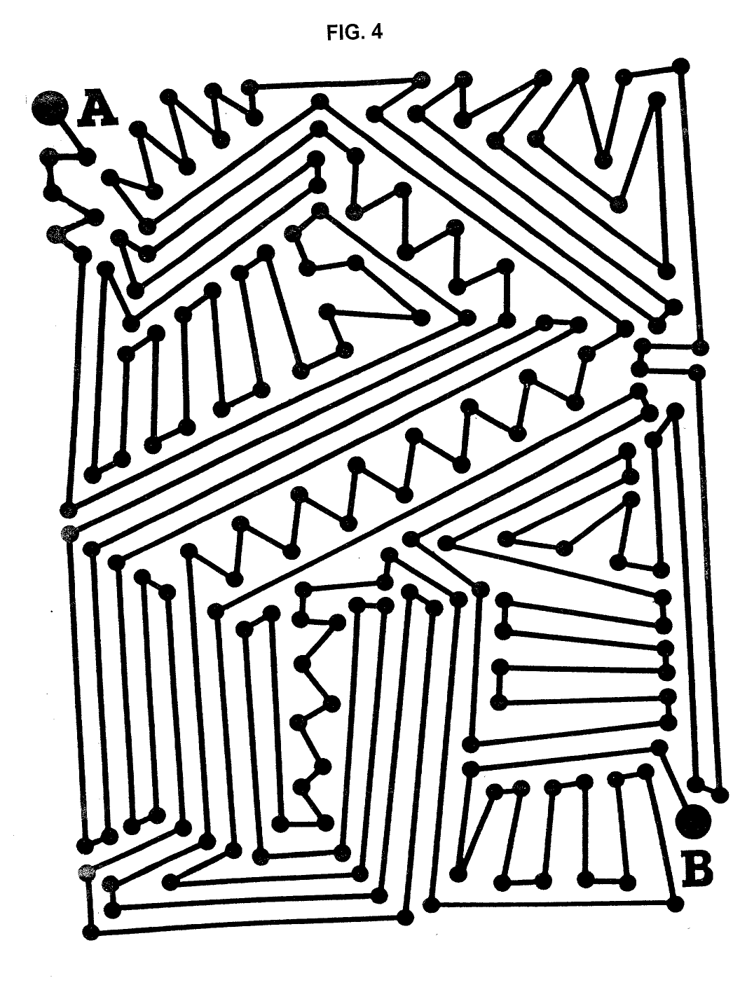

Some of the individual exercises in the exercise sequences use charts. For example, the steps of Fusion and Fusion Pumping use a Fusion Chart, such as that shown in FIG. 2. FIG. 3 is a Word Chart that is scanned in, for example, the Word Chart exercise. The Scanning Chart of FIG. 4 is used with, for example, the Scanning exercise.

Each individual sequence is itself useful in exercising the muscles in and around the eye. In a preferred embodiment, at least one of General Sequences A through G, preferably at least one of General Sequences A through D, is performed approximately each day. More preferably, at least one of Timed Sequences H through N, preferably at least one of Timed Sequences H through K, is performed approximately each day. Most preferably, at least one of Ordered Sequences O through U, preferably at least one of Ordered Sequences o through R, is performed approximately each day.

As an option, two exercise sequences may be performed in approximately a single day. Performing two exercise sequences per approximate day has been found to have a synergistic effect, referred to as a “potentiation effect,” which causes a more rapid improvement. As an option, in addition to performing at least one exercise sequence, an eye patch may be worn for at least approximately one hour each approximate day.

Although strengthening and toning the eye muscles, improvement of coordination of the eye muscles, improvement of the flow of blood and other fluids in and around the eyes and improvement in vision are accomplished when at least one exercise sequence is performed each approximate day, it is preferred that General Sequences A through D or Timed Sequences H through K are performed on a rotating basis. More preferably, Ordered Sequences O through R are performed on a rotating basis. For example, General Sequence A (or Timed Sequence H or Ordered Sequence O) may be performed on day one, General Sequence B (or Timed Sequence I or Ordered Sequence P) on day two, General Sequence C (or Timed Sequence J or Ordered Sequence Q) on day three, General Sequence D (or Timed Sequence K or Ordered Sequence R) on day four, and repeat the sequence. Alternatively, General Sequences A and B (or Timed Sequences H and I or Ordered Sequences O and P) may be performed on day one, General Sequences C and D (or Timed Sequences J and K or Ordered Sequences Q and R) on day two, General Sequences A and B (or Timed Sequences H and I or Ordered Sequences O and P) on day three, etc. By performing four exercise sequences in order, more rapid and complete improvement has been observed.

In addition to the general benefits obtained by practicing the sequence of the invention on a daily basis, the inventors have found that practicing particular sets of sequences on a regular basis are particularly useful for counteracting Computer Vision Syndrome. In particular, General Sequences E through G, or Timed Sequences L through N, or Ordered Sequences S through U, are useful in treating or preventing the effects of Computer Vision Syndrome. Preferably, General Sequences E through G are performed on a rotating basis, preferably by performing one of the General Sequences E through G, more preferably, the Timed Sequences L through N and most preferably the Ordered Sequences S through U. In any of these embodiments, the sequences are preferably performed approximately periodically during times of activities which may lead to Computer Vision Syndrome. For example, sequences may be performed at approximately periodic intervals of about hour or less. Preferably, sequences are performed at approximately periodic intervals of about one hour. For example, in preferred embodiments, during computer use or any other activity that may lead to Computer Vision Syndrome, one of the eye relaxation sets, i.e. General Sequence E, Timed Sequence L or Ordered Sequence S, is practiced after beginning computer use. At some later time, for example approximately one hour later, one of the eye massage sets, i.e. General Sequence F, Timed Sequence M or Ordered Sequence T is performed, followed by one of the power vision sets, i.e. General Sequence G, Timed Sequence N, or Ordered Sequence U at some later time, for example approximately one hour later. Although this embodiment rotates the eye relaxation set sequences followed by the eye massage set sequences followed by the power vision set sequences, it is also advantageous to perform these three sets of sequences in different order or at different intervals.

Although practicing the above sets and sequences has a positive effect on strengthening the muscles in and around the eye to treat or prevent Computer Vision Syndrome, the results are further improved by adding additional “visual habits” at intervals interspersed between the time when the individual sequences are performed. Examples of visual habits include looking away from the computer and focusing on an object at least approximately ten feet away, and doing a slow blink from the slow blinking exercise. In a particularly preferred embodiment, looking away from the computer or performing a slow blink are performed at time intervals approximately equally interspersed between practicing the sequences.

The present invention also includes a kit having materials for teaching the exercises and sequences and assisting in practicing the exercises and sequences. Because of the visual nature of the invention, the kit preferably comprises a presentation. The presentation may be, for example, an instruction manual, one or more videotapes and charts used to practice the invention. The instructions for practicing the sequences may be contained in an instruction manual, one or more videotapes, or both. The presentation may further include at least one of one or more audio tapes, a journal for tracking progress of the person practicing the method and an eye patch.

Examples of the materials identified above and others that may be provided in a kit include: an instruction manual that may include a welcome letter, illustrations and cover materials incorporated in the instruction manual or supplied separately; descriptions of Acupressure Variation A, Acupressure Variation B, Acupressure Variation C, and Acupressure Variation D; charts used in practicing the exercises of the invention; a quick map with illustrated summaries of exercises that comprise sequences of the invention; a daily journal for recording activities and progress in practicing the invention; a daily reminder page; an exercise card with summaries of exercises that comprise sequences of the invention; and a videotape for use with the invention. Instead of a videotape, a video medium can be used to store a video presentation for teaching the exercise steps. As an example, a video medium can be used to produce a video image on a television, a video monitor, or a computer. A video medium can be, for example, a videotape, a laser disc, an optical disc, a video CD, a CD-ROM, a DVD, or a computer-recordable medium.

In another embodiment of invention, the presentation is embodied on a computer-readable medium. A “computer-readable medium” refers to any storage device used for storing data accessible by a computer. Examples of a computer-readable medium include: a magnetic hard disk; a floppy disk; an optical disk, such as a CD-ROM and a DVD; a magnetic tape; a memory chip; and a carrier wave used to carry computer-readable electronic data, such as those used in transmitting and receiving e-mail and in accessing a network.

A “computer” refers to any apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a microcomputer; a personal computer (PC); a serial-processing computer; a parallel-processing computer; a server; an interactive television; and a hybrid combination of a computer and an interactive television. A computer also refers to two or more computers connected together via a network for transmitting or receiving information between the computers. An example of such a computer includes a distributed computer system for processing information via computers linked by a network.

“Software” refers to prescribed rules to operate a computer. Examples of software include: software; code segments; instructions; computer programs; and programmed logic.

A “network” refers to a number of computers and associated devices that are connected by communication facilities. A network involves permanent connections such as cables or temporary connections such as those made through telephone or other communication links. Examples of a network include: the Internet; a local area network (LAN); and a wide area network (WAN).

In yet another embodiment of the invention, a presentation of the exercises of the invention is accessed via a web site on the Internet. A user accesses the web site and gains entry to restricted areas with information, instructions, presentations and charts for practicing the invention. The information can then be downloaded to the user's computer and printed. As an option, access to the web site may be limited to users who obtain or purchase rights to practice the invention.

As an option, the Internet may be used in practicing other embodiments of the invention. A user may access a web site that contains additional exercises or information to the user useful in practicing the invention. For example, if improvements to the invention are made by the development of additional sequences of exercises which are found to be useful, a user may receive these instructions at the web site. As an option, the web site can have portions accessible only to purchasers of the kit of the invention, who would then have access to future developments via the web site.

In another embodiment of the invention, a presentation of the exercises of the invention is embodied in a seminar. In the seminar, instructions are presented for performing one or more of the sequences of the invention.

In another embodiment of the invention, instructions are presented for performing one or more of the sequences of the invention. Examples of techniques for presenting instructions include: an instruction manual, a web site, a seminar, a one-on-one lesson, a classroom-type lesson, a computer-readable medium, a kit, a chart, a journal, a card, a daily reminder page, a videotape, an audio tape, a closed circuit or television broadcast and a book.

In another embodiment of the invention, a reminder implemented with software reminds a user of a computer to perform one or more exercises or sequences of the invention. This reminder may repeat at specific intervals, for example, approximately once per hour.

In yet another embodiment of the invention, the sets of sequences described above for counteracting the affects of Computer Vision Syndrome, i.e. General Sequences E to G, Timed Sequences L to N or Ordered Sequences S to U, are contained on a computer-readable medium. For example, such a medium may include instructions for performing the exercises as well as including the precise exercise sequences embodied on a computer-readable format.

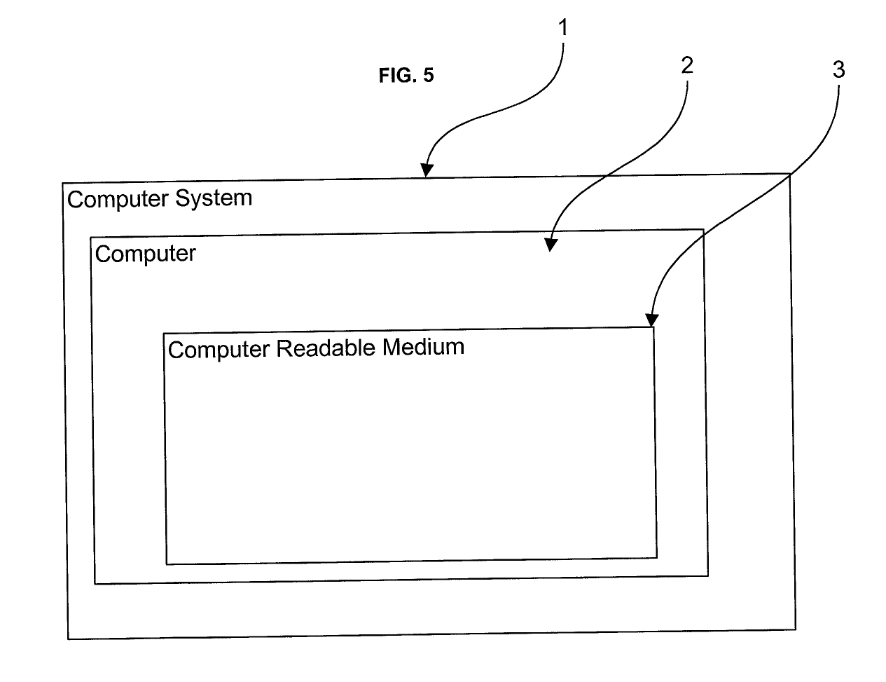

In a particularly preferred embodiment, the computer-readable medium contains software to be used with a computer system. FIG. 5 is a schematic of the computer system of the invention. The computer system 1 includes a computer 2 which further includes a computer-readable medium 3 having software. The software provides instructions for performing the exercises and sequences particularly applicable to preventing and/or treating Computer Vision Syndrome, i.e. General Sequences E to G, Timed Sequences L to N or Ordered Sequences S to U. The software presents instructions which preferably appear on a computer monitor of the computer system 1. Preferably, the software presents the instructions at approximately one hour intervals or at other greater or shorter times which may be determined by the user. The instructions may be preceded by a signal, such as an audio and/or a video signal, indicating that it is time to perform a sequence and indicating which sequence to perform. As an option, instructions for performing other sequences of the invention may be provided by software embodied on a computer-readable medium.

Thus, for example, the program may signal the computer system user to perform a particular sequence and present instructions on the computer monitor for performing a sequence, for example General Sequence E, Timed Sequence L or Ordered Sequence S. Approximately one hour later, the computer may again produce a signal accompanied by instructions on how to perform the next sequence, for example General Sequence F, Timed Sequence M or Ordered Sequence T. After approximately one more hour, the computer may signal the user to perform a third sequence, for example General Sequence G, Timed Sequence N or Ordered Sequence U. Once this cycle of exercises is complete, the computer presents a signal approximately one hour later for the user to perform the first set of exercises and this sequence continues. Although the sequential order of performing the sequences described above has been found to be particularly useful, the sequences may be performed in any order and at different regular or irregular intervals.

In a particularly preferred embodiment, in addition to approximately periodic signals to perform one of the sequences designed to treat or prevent Computer Vision Syndrome, a software implemented signal may remind the user, at times intermediate to performing the sequences, to perform the visual habits. For example, at some time after signaling the user to perform the first exercise sequence, such as, for example approximately twenty minutes later, the computer may signal the user to practice one of the visual habits. The computer may present a second signal to perform one of the visual habits prior to signaling to perform the next sequence. For example, approximately twenty minutes after signaling to perform the first visual habit, or at some other time before performing the second set of sequences, the computer may signal the user to perform the same or a different visual habit. This cycle continues such that, in between any two exercise sequences, there are instructions to perform the steps associated with at least one visual habit. In another example, signaling to perform the visual habits may occur more frequently, for example approximately every three minutes. More frequent performance of the visual habits may be particularly beneficial for severe symptoms, for example, severe or migraine headaches.

Although this particularly preferred embodiment includes signaling to perform two of the visual habits between two exercise sequences, it will be appreciated that this is optional and any number of visual habits may be performed between two sequences including none, one or more than two. Further, the intervals between the visual habits may be unequal. Also, although this particularly preferred embodiment has been described with respect use of a computer system, a computer system is not required.

In an even more preferred embodiment of the invention, the software may allow the user to modify instructions for performing a sequence by adding, deleting or changing individual exercises within a sequence initially presented by the software. Thus, during use, a user who finds that a sequence other than the initially present sequences (such as General Sequences E through G, Timed Sequences L through N or Ordered Sequences S through U) is beneficial, or determines that interspersing additional exercises within a sequence is beneficial, the software can be modified by the user and subsequently present instructions for performing the modified sequences. Thus, for example, if a particular user finds that, for example, General Sequence A is beneficial to that user, the software may be modified to present instructions for performing General Sequence A in addition to or in place of one of the sequences initially present. Also, the software may preferably be modified to prompt the user to perform General Sequence A at some predetermined time during the approximate day, or as part of the general rotation of sequences performed approximately periodically and signaled by the software implemented reminder. It will be appreciated that the substituted and modified sequences are not limited to the sequences described herein, but may include any other sequence created by the user, particularly those created by modifying the original sequences, or presented on the web site previously described. Thus, a wide range of sequences, including sequences described herein and sequences described in the prior art, developed individually by the user, or developed by the inventor or others may be incorporated into software on a computer-readable medium and that may be used in a computer system. In the process for modifying or adding sequences, the software may automatically determine the time over which an exercise or sequence is performed or the user may determine the time. For example, the software may set the time for performing added exercises to approximately forty seconds.

The invention includes sequences to assist the general population as well as particular segments of the population, namely, those having Computer Vision Syndrome. Other sequences may be developed to assist other sections of the population, for example, the elderly, children, avid book readers, hunters and any other segment where eye strain or exhaustion may occur. In addition, embodiments of the invention may be varied to address the needs of particular segments of the population. For example, computer games may comprise computer-readable media comprising software of the invention. This embodiment may be especially useful for presenting eye exercises to children. Other embodiments are well within the skill of persons skilled in the art.

The embodiments illustrated and discussed herein are intended only to teach those skilled in the art the best way known to the inventors to make and use the invention. Nothing in this specification should be considered as limiting the scope of the present invention. The above-described embodiments of the invention may be modified or varied, and elements added or omitted, without departing from the invention, as appreciated by those skilled in the art in light of the teachings herein. It is therefore to be understood that, within the scope of the claims and their equivalents, the invention may be practiced otherwise than as specifically described.

What is claimed is:

1. A kit for performing a method for exercising eye muscles comprising:
a presentation comprising presenting at least one sequence selected from the group consisting of General Sequence A, General Sequence B, General Sequence C, General Sequence D, General Sequence E, General Sequence F, General Sequence G, Timed Sequence H, Timed Sequence I, Timed Sequence J, Timed Sequence K, Timed Sequence L, Timed Sequence M, Timed Sequence N, Ordered Sequence O, Ordered Sequence P, Ordered Sequence Q, Ordered Sequence R, Ordered Sequence S, Ordered Sequence T, and Ordered Sequence U.

2. The kit of claim 1, further comprising an eye patch.

3. The kit of claim 1, wherein said at least one sequence is selected from the group consisting of General Sequence A, General Sequence B, General Sequence C, General Sequence D, Timed Sequence L, Timed Sequence M, Timed Sequence N, Ordered Sequence O, Ordered Sequence P, Ordered Sequence Q, and Ordered Sequence R and performed in approximately a day.

4. The kit of claim 1, wherein said at lest one sequence is selected from the group consisting of General Sequence E, General Sequence F, General Sequence G, Timed Sequence H, Timed Sequence I, Timed Sequence J, Timed Sequence K, Ordered Sequence S, Ordered Sequence T, and Ordered Sequence U and performed in approximately an hour.

5. The kit of claim 1, wherein said at least one sequence comprises General Sequence A.

6. The kit of claim 1, wherein said at least one sequence comprises General Sequence B.

7. The kit of claim 1, wherein said at least one sequence comprises General Sequence C.

8. The kit of claim 1, wherein said at least one sequence comprises General Sequence D.

9. The kit of claim 1, wherein said at least one sequence comprises General Sequence E.

10. The kit of claim 1, wherein said at least one sequence comprises General Sequence F.

11. The kit of claim 1, wherein said at least one sequence comprises General Sequence G.

12. The kit of claim 1, wherein said at least one sequence comprises Timed Sequence H.

13. The kit of claim 1, wherein said at least one sequence comprises Timed Sequence I.

14. The kit of claim 1, wherein said at least one sequence comprises Timed Sequence J.

15. The kit of claim 1, wherein said at least one sequence comprises Timed Sequence K.

16. The kit of claim 1, wherein said at least one sequence comprises Timed Sequence L.

17. The kit of claim 1, wherein said at least one sequence comprises Timed Sequence M.

18. The kit of claim 1, wherein said at least one sequence comprises Timed Sequence N.

19. The kit of claim 1, wherein said at least one sequence comprises Ordered Sequence O.

20. The kit of claim 1, wherein said at least one sequence comprises Ordered Sequence P.

21. The kit of claim 1, wherein said at least one sequence comprises Ordered Sequence Q.

22. The kit of claim 1, wherein said at least one sequence comprises Ordered Sequence R.

23. The kit of claim 1, wherein said at least one sequence comprises Ordered Sequence S.

24. The kit of claim 1, wherein said at least one sequence comprises Ordered Sequence T.

25. The kit of claim 1, wherein said at least one sequence comprises Ordered Sequence U.

26. The kit of claim 1, wherein said presenting at least one sequence comprises presenting at least
General Sequence A,
General Sequence B,
General Sequence C, and
General Sequence D.

27. The kit of claim 1, wherein said presenting at least one sequence comprises presenting at least
General Sequence E,
General Sequence F, and
General Sequence G.

28. The kit of claim 1, wherein said presenting at least one sequence comprises presenting at least
Timed Sequence H,
Timed Sequence G,
Timed Sequence J, and
Timed Sequence K.

29. The kit of claim 1, wherein said presenting at least one sequence comprises presenting at least
Timed Sequence L,
Timed Sequence M, and
Timed Sequence N.

30. The kit of claim 1, wherein said presenting at least one sequence comprises presenting at least
Ordered Sequence O,
Ordered Sequence P,
Ordered Sequence Q, and
Ordered Sequence R.

31. The kit of claim 1, wherein said presenting at least one sequence comprises presenting at least
Ordered Sequence S,
Ordered Sequence T, and
Ordered Sequence U.

32. The kit of claim 1, wherein said presenting at least one sequence comprises presenting at least
General Sequence A, followed by
General Sequence B, followed by
General Sequence C, and followed by
General Sequence D.

33. The kit of claim 1, wherein said presenting at least one sequence comprises presenting at least
General Sequence E, followed by
General Sequence F, followed by
General Sequence G.

34. The kit of claim 1, wherein said presenting at least one sequence comprises presenting at least
Timed Sequence H, followed by
Timed Sequence I, followed by
Timed Sequence J, followed by
Timed Sequence K.

35. The kit of claim 1, wherein said presenting at least one sequence comprises presenting at least
Timed Sequence L, followed by
Timed Sequence M, followed by
Timed Sequence N.

36. The kit of claim 1, wherein said presenting at least one sequence comprises presenting at least
Ordered Sequence O, followed by
Ordered Sequence P, followed by
Ordered Sequence Q, followed by
Ordered Sequence R.

37. The kit of claim 1, wherein said presenting at least one sequence comprises presenting at least
Ordered Sequence S, followed by
Ordered Sequence T, followed by
Ordered Sequence U.

38. The kit of claim 1, wherein said presentation comprises an instruction manual.

39. The kit of claim 1, wherein said presentation comprises a video tape.

40. The kit of claim 1, wherein said presentation comprises an audio tape.

41. The kit of claim 1, wherein said presentation comprises a computer-readable medium.

42. The kit of claim 1, further comprising at least one visual chart corresponding to at least one of General Sequence A, General Sequence B, General Sequence C, General Sequence D, General Sequence E, General Sequence F, General Sequence G, Timed Sequence H, Timed Sequence I, Timed Sequence J, Timed Sequence K, Timed Sequence L, Timed Sequence M, Timed Sequence N, Ordered Sequence O, Ordered Sequence P, Ordered Sequence Q, Ordered Sequence R, Ordered Sequence S, Ordered Sequence T, and Ordered Sequence U; a journal for recording completion of steps and tracking progress; a word chart; a fusion chart; and a scanning chart.

43. The kit of claim 1, wherein the presentation further comprises a software-implemented periodic reminder embodied on a computer-readable medium.

44. A method of presenting an exercise program for exercising eye muscles comprising the steps of:
providing the kit of claim 1; and
providing a web site accessible via the Internet comprising instructions for performing at least one exercise for exercising the eye muscles.

45. A method of presenting an exercise program for exercising eye muscles comprising the step of:
presenting at least one sequence selected from the group consisting of General Sequence A, General Sequence B, General Sequence C, General Sequence D, General Sequence E, General Sequence F, General Sequence G, Timed Sequence H, Timed Sequence I, Timed Sequence J, Timed Sequence K, Timed Sequence L, Timed Sequence M, Timed Sequence N, Ordered Sequence O, Ordered Sequence P, Ordered Sequence Q, Ordered Sequence R, Ordered Sequence S, Ordered Sequence T, Ordered Sequence U.

46. The method of claim 45, wherein said presenting at least one sequence comprises providing a web site accessible via the Internet.

47. The method of claim 45, wherein said presenting at least one sequence comprises a seminar.

48. A computer system comprising a computer-readable medium having software, the software comprising:
software for presenting at least one sequence selected from the group consisting of General Sequence A, General Sequence B, General Sequence C, General Sequence D, General Sequence E, General Sequence F, General Sequence G, Timed Sequence H, Timed Sequence I, Timed Sequence J, Timed Sequence K, Timed Sequence L, Timed Sequence M, Timed Sequence N, Ordered Sequence O, Ordered Sequence P, Ordered Sequence Q, Ordered Sequence R, Ordered Sequence S, Ordered Sequence T, and Ordered Sequence U.

49. The computer system of claim 48, further comprising:
software for modifying at least one sequence selected from the group consisting of General Sequence A, General Sequence B, General Sequence C, General Sequence D, General Sequence E, General Sequence F, General Sequence G, Timed Sequence H, Timed Sequence I, Timed Sequence J, Timed Sequence K, Timed Sequence L, Timed Sequence M, Timed Sequence N, Ordered Sequence O, Ordered Sequence P, Ordered Sequence Q, Ordered Sequence R, Ordered Sequence S, Ordered Sequence T, and Ordered Sequence U, by adding, substituting or deleting at least one eye exercise to obtain at least one modified sequence; and
software for presenting instructions for performing said at least one modified sequence.

50. The computer system of claim 48, further comprising
software for presenting a reminder to perform at least one eye exercise or sequence.

51. The computer system of claim 48, further comprising:
software for presenting one or more eye exercises selected from the group consisting of Pumping, Tromboning, Clock Rotations, Eye Rolls, Slow, Fast and Squeeze Blinking, Blur Zoning, Nose Fusion, use of a Fusion Chart, Fusion Pumping, Blur Reading, Scanning a Scanning Chart, using a Word Chart, Acupressure selected from the group consisting of Acupressure Variation A, Acupressure Variation B, Acupressure Variation C and Acupressure Variation D, Light Therapy, Palming, and Hydrotherapy.

52. A computer system comprising a computer-readable medium having software, the software comprising: software for presenting at least one sequence selected from the group consisting of General Sequence E, General Sequence F, General Sequence G, Timed Sequence L, Timed Sequence M, Timed Sequence N, Ordered Sequence S, Ordered Sequence T, and Ordered Sequence U.

53. The computer system of claim 52, wherein said computer system further comprises software for presenting a reminder to perform at least one sequence selected from the group consisting of General Sequence E, General Sequence F, General Sequence, Timed Sequence L, Timed Sequence M, Timed Sequence N, Ordered Sequence S, Ordered Sequence T, and Ordered Sequence U.

54. The computer system of claim 53, wherein said reminder is presented approximately once each hour.

55. The computer system of claim 52, further comprising:
software for modifying at least one sequence selected from the group consisting of General Sequence E, General Sequence F, General Sequence G, Timed Sequence L, Timed Sequence M, Timed Sequence N, Ordered Sequence S, Ordered Sequence T, and Ordered Sequence U by adding, substituting or deleting at least one eye exercise to obtain at least one modified sequence; and
software for presenting instructions for performing said at least one of modified sequence.

56. The computer system of claim 52, further comprising
software for presenting at least one visual habit selected from the group consisting of focusing on an object at least ten feet away and slow blinking.

57. The computer system of claim 52, further comprising software for presenting a reminder to practice at least one visual habit.

58. The computer system of claim 57, wherein said visual habit is selected from the group consisting of focusing on a distant object and slow blinking.

59. A method for exercising eye muscles, comprising the step of:
practicing at least one sequence selected from the group consisting of General Sequence A, General Sequence B, General Sequence C, General Sequence D, General Sequence E, General Sequence F, General Sequence G, Timed Sequence H, Timed Sequence I, Timed Sequence J, Timed Sequence K, Timed Sequence L, Timed Sequence M, Timed Sequence N, Ordered Sequence O, Ordered Sequence P, Ordered Sequence Q, Ordered Sequence R, Ordered Sequence S, Ordered Sequence T, and Ordered Sequence U.

60. The method of claim 59, further comprising wearing an eye patch for at least approximately one hour in an approximate day.

61. The method of claim 59, wherein said practicing at least one sequence comprises practicing a sequence set selected from the group consisting of Group (I), Group (II), and Group (III), Group (IV), Group (V) and Group (VI), wherein
Group (I) comprises General Sequence A, practicing General Sequence B, practicing General Sequence C; and practicing General Sequence D;
Group (II) comprises Timed Sequence H, Timed Sequence I, Timed Sequence J, and Timed Sequence K;
Group (III) comprises Ordered Sequence O, Ordered Sequence P, Ordered Sequence Q, and Ordered Sequence R; pg,33
Group (IV) comprises General Sequence A, followed by General Sequence B, followed by General Sequence C, followed by General Sequence D;
Group (V) comprises Timed Sequence H, followed by Timed Sequence I, followed by Timed Sequence J, followed by Timed Sequence K; and
Group (VI) comprises Ordered Sequence O, followed by Ordered Sequence P, followed by Ordered Sequence Q, followed by Ordered Sequence R.

62. The method of claim 61, wherein at least one sequence is practiced within approximately one day.

63. The method of claim 59, wherein said practicing at least one sequence comprises practicing a sequence set selected from the group consisting of Group (X), Group (XI), and Group (XII), Group (XIII), Group (XIV) and Group (XV); wherein
Group (X) comprises General Sequence E, practicing General Sequence F; and practicing General Sequence G;
Group (XI) comprises Timed Sequence L, Timed Sequence M, and Timed Sequence N;
Group (XII) comprises Ordered Sequence S, Ordered Sequence T, and Ordered Sequence U;
Group (XIII) comprises General Sequence E, followed by General Sequence F, followed by General Sequence G;
Group (XIV) comprises Timed Sequence L, followed by Timed Sequence M, followed by Timed Sequence N; and
Group (XV) comprises Ordered Sequence S, followed by Ordered Sequence T, followed by Ordered Sequence U.

64. The method of claim 63, wherein at least one sequence is practiced within approximately one hour.

65. The method of claim 59, further comprising practicing at least one visual habit.
